(19) United States
(12) Patent Application Publication
(10) Pub. No.: US 2025/0115939 A1
(43) Pub. Date: Apr. 10, 2025

(54) Neural Repair Protein Composition and Preparation Method thereof and Use thereof

(71) Applicant: BEIJING DARWIN BIOTECH CO., LTD., Beijing (CN)

(72) Inventors: Yu WANG, Beijing (CN); Wenyong GAO, Beijing (CN); Lin CHEN, Beijing (CN); Jianjun LI, Beijing (CN)

(21) Appl. No.: 18/833,908

(22) PCT Filed: Jan. 28, 2023
(86) PCT No.: PCT/CN2023/073582

§ 371 Date: Jul. 28, 2024

(30) Foreign Application Priority Data
Jan. 28, 2022 (CN) 202210105729.X
Apr. 2, 2022 (CN) 202210351455.2
Jul. 5, 2022 (CN) 202210793711.3

(51) Int. Cl. C12P 21/00; A61K 38/02; C07K 1/16; C12N 5/0775

(52) U.S. Cl. CPC C12P 21/00; A61K 38/02; C07K 1/16; C12N 5/0662; C12N 2500/25; C12N 2500/32; C12N 2501/11; C12N 2501/113; C12N 2501/999

(57) ABSTRACT

The present disclosure relates to a neural repair protein composition. The preparation thereof includes the following steps: adding 20 U/mL-35 U/mL of any one of or a combination of nuclease or omnipotent nuclease to a cell protein extract, performing enzymatic hydrolysis at 37° C.±1° C. for 15-40 minutes, and separating and purifying the prepared enzymatic hydrolysate. The neural repair protein composition of the present disclosure has the effects of cell repair and nerve damage repair, and can be used to repair nerve damage caused by diseases such as central nervous system damage, neurodegenerative diseases, stroke, brain damage, ataxia, cerebral hemorrhage, Alzheimer's disease, Parkinson's disease, senile dementia or complications thereof. It has the advantages of good stability, high bioavailability, safety and effectiveness, and is easy to produce and store.

### TECHNICAL FIELD

The present disclosure belongs to the field of biopharmaceutical technology and specifically relates to a neural repair protein composition, preparation method thereof, and use thereof.

### BACKGROUND

Cells are the fundamental unit of life activities and the foundation of body health. When the redox balance of the body is disrupted, it can cause an interruption in redox signaling and control, leading to oxidative stress damage and various diseases. Cerebral ischemia and hypoxia (such as stroke, cerebral hemorrhage, long-term high-altitude work, occlusion and/or stenosis of the internal carotid and vertebral arteries, etc.) and oxidative stress damage can cause various neurodegenerative diseases caused by the loss of neurons or glial cells, including memory loss, Alzheimer's disease, Parkinson's disease, senile dementia, multiple sclerosis, ataxia, Huntington's disease, amyotrophic lateral sclerosis, etc. At present, there are more than 3 million Parkinson's patients, more than 200 thousand amyotrophic lateral sclerosis patients, and more than 10 million people with senile dementia in China. With the increasing aging of the global population, there are high incidence rate and other characteristics. Timely repair of damaged cells can significantly improve and treat related lesions.

Stroke (stenosis or occlusion of cerebral blood supply arteries resulting in the inability of blood to flow into the brain) and cerebral hemorrhage (sudden rupture of cerebral blood vessels) are common central nervous system diseases. The ischemic and hypoxic damage of brain tissue is caused by insufficient cerebral blood supply, and it shows corresponding neurological deficit symptoms, including balance problems, hemiplegia, loss of sensation and vibration sensation, numbness, weak reflexes, ptosis, visual field defects, aphasia and loss of use. Excessive free radicals are the main reason for the aggravation of ischemia reperfusion injury in early acute ischemic stroke. This disease is characterized by high incidence rate, poor disease prognosis, high recurrence rate, high disability rate, and high mortality, which seriously affects human health and quality of life. Ataxia is often caused by dysfunction of the cerebellum, proprioception, and vestibule, resulting in clumsiness and disharmony in movement. When it affects the trunk, limbs, and pharyngeal muscles, it can cause balance, posture, gait, and speech disorders, including cerebellar ataxia, cerebral ataxia, sensory ataxia, vestibular ataxia, etc. The etiology is complex and often a comprehensive manifestation of various diseases, including acute brain swelling caused by infarction, edema, or bleeding. Brain trauma is often caused by external objects in the brain, often leading to varying degrees of permanent functional impairment, including abnormal focal symptoms in areas such as movement, sensation, speech, vision, and hearing. Peripheral nerve damage is a common clinical disease mainly caused by trauma, tumor, metabolic disease (such as diabetes and its complications) and other factors, which often leads to partial or complete loss of motor, sensory and autonomic functions of the affected segments of the body, and even intractable neuralgia, seriously affecting the quality of life of patients. The World diabetes Report released by WHO in 2017 shows that the number of diabetes patients worldwide is increasing year by year. There are 129 million patients with diabetes in China, and its various complications (including neuralgia and nerve injury caused by ischemia and hypoxia of peripheral and peripheral nerves) lead to disability and death of diabetes patients and other serious consequences.

The clinical treatment of stroke emphasizes both thrombolysis and neuroprotection to reverse neuronal apoptosis and improve nerve damage. Butylphthalein can reduce intracellular calcium ion concentration, inhibit glutamate release and reduce arachidonic acid production, eliminate oxygen free radicals, and improve oxidase activity. It acts on multiple pathological stages of cerebral ischemic lesions and is clinically used to improve local circulation, reduce infarct area, alleviate brain tissue damage, and restore neurological function. It is mainly used to improve neurological deficits in patients with acute ischemic stroke, and is mainly used for the treatment of mild to moderate acute ischemic stroke. Edaravone camphor is an antioxidant and free radical scavenger that can eliminate various free radicals, reduce brain edema and tissue damage, inhibit the expression of inflammatory factors caused by cerebral ischemia-reperfusion, and reduce cell apoptosis and necrosis. It is clinically used to improve neurological symptoms, daily living ability, and functional impairment caused by acute cerebral infarction.

When cells and microorganisms are subjected to external stimuli and external stressors (including cold, heat, acid, alkali, current, radiation, chemicals, etc.), they will be induced to produce stress proteins due to stress responses. Reference 1 (New limonophyllines A-C from the stem of Atalantia monophylla and cytotoxicity against cholangiocarcinoma and HepG2 cell lines, Arch. Pharm. Res. (2018) 41:431-437) reveals that compounds 1-16 extracted from the Rutaceae plant (Atalantia monophylla) exhibiting activities such as inhibiting tumor cell growth.

Mesenchymal stem cells (MSCs) have the potential for self-replication and multi-lineage differentiation, and they are widely present in tissues such as bone marrow, fat, synovium, dental pulp, amniotic fluid, placenta, umbilical cord, embryo, umbilical cord blood, amniotic membrane, peripheral blood, muscle, urine, etc. They have the characteristics of wide sources, no need for matching, low infection rate, strong differentiation potential, strong proliferation ability, and easy collection. They can produce active factors such as stem cell growth factor (SCF), nerve growth factor (NGF), interleukin-6 (IL-6), interleukin-7 (IL-7), tumor necrosis factor (TNF), interferon (IFN), which involve in regulating cell growth, apoptosis, differentiation, antiviral therapy, immune maturation, and other processes, and are used for immune regulation, tissue repair, and the treatment of acute lung damage, severe pneumonia, acute respiratory distress syndrome, and other diseases. However, MSCs products need to be refrigerated in their production, storage, transportation, and application processes, and their cell viability should be maintained for ≤12 hours, which limits their therapeutic applications. Therefore, it is necessary to develop safe and effective nerve repair drugs to meet clinical needs.

### SUMMARY

The purpose of the present disclosure is to provide a neural repair protein composition, comprising the following steps for preparation:
(1) adding 20 U/mL-35 U/mL of any one of or a combination of nuclease or omnipotent nuclease to a cell protein extract, and performing enzymatic hydrolysis at 37° C.±1° C. for 15-40 minutes to obtain an enzymatic hydrolysate;
(2) under conditions of 2° C.-8° C., preparing the enzymatic hydrolysate obtained in step (1) to a 5-15 mg/ml solution with an eluent, and then passing through a chromatographic column with an eluent flow rate of 0.1-1 mL/min, monitoring and collecting an eluate fraction with a UV wavelength of 280 nm, wherein the eluent solvent consists of 50 mmol/L phosphate buffer (pH 6.8) containing 300 mmol/L sodium chloride.

In a preferred technical solution of the present disclosure, the preparation of the protein extract includes the following steps:
S-1: placing mesenchymal passage cells with a density of 5.0×10⁶ cells/mL to 1.0×10⁷ cells/mL in a culture medium containing DMEM/F12 40-50%, RPMI1640 40-50%, bovine serum albumin (BSA) 0.1-2%, epidermal growth factor (EGF) 1-15 μg/mL, fibroblast growth factor (FGF) 1-15 μg/mL, insulin transferrin 1-15 μg/mL, compound amino acids (18AA) 0.01-0.1%, and 2-10 μmol/L of a stressor, and then culture it under conditions of 37.0° C.±0.5° C. and 5%±1.0% CO₂ for 2 to 6 hours and then performing isolation, washing, and collecting cells, wherein the stressor is selected from any one of compounds 1-16 or a combination thereof;

| Compounds | General formula | Substituents |
|---|---|---|
| 1 2 3 | 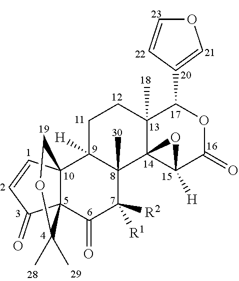 | R¹ = OCH₃, R² = OH R¹ = R² = OH R¹ = OH, R² = H |
| 4 5 | 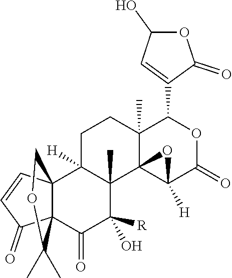 | R = H R = OH |
| 6 7 8 | 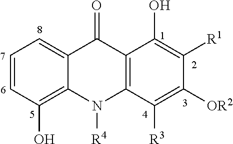 | R¹ = prenyl, R² = H, R₃ = prenyl, R⁴ = CH₃ R¹ = prenyl, R² = H, R₃ = prenyl, R⁴ = H R¹ = H, R² = H, R₃ = OCH₃, R⁴ = CH₃ |
| 9 | | R¹ = H, R² = CH₃, R₃ = OCH₃, R⁴ = CH₃ |
| 10 | | R¹ = OCH₃, R² = H, R₃ = H, R⁴ = CH₃ |
| 11 | | R¹ = OCH₃, R² = H, R₃- OCH₃, |
| | | R⁴ = CH₃ |
| 12 | | R¹ = prenyl, R² = CH₃, R₃ = H, R⁴ = H |
| 13 14 | 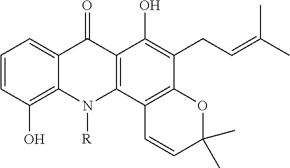 | R = H R = CH₃ |
| 15 | 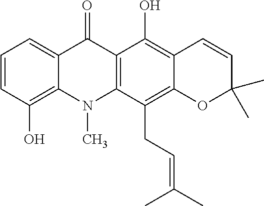 [SMILES: CC(C)=CCc1c2c(c(O)c3c(=O)c4cccc(O)c4n(C)c13)C=CC(C)(C)O2] | |
| 16 | 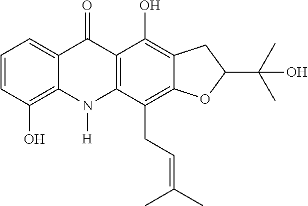 [SMILES: CC(C)=CCc1c2c(c(O)c3c(=O)c4cccc(O)c4[nH]c13)CC(C(C)(C)O)O2] | |

S-2: dispersing the collected cells in a solvent at a density of 5.0×10⁶ cells/mL-5.0×10⁷ cells/mL, and then performing an ultrasonic treatment on the collected cells at 2° C.-8° C. to prepare a cell lysate, wherein the solvent is selected from any one or a combination of physiological saline, 5% glucose solution, phosphate buffer solution (PBS), TBPS buffer, TBST buffer, or Tris buffer;
S-3: separating the cell lysate prepared in step S-2, the then sequentially filtering a separated solution through 0.45 μm, 0.22 μm filter membranes, thereby obtaining the cell protein extract.

In a preferred technical solution of the present disclosure, the culture medium in step S-1 contains DMEM/F12 42-45%, RPMI1640 42-45%, bovine serum albumin (BSA) 0.5-1.5%, epidermal growth factor (EGF) 5-10 μg/mL, fibroblast growth factor (FGF) 5-10 μg/mL, insulin transferrin 5-10 μg/mL, compound amino acids (18AA) 0.02-0.05%, and 3-8 μmol/L of stressor.

In a preferred technical solution of the present disclosure, the culture medium in step S-1 contains DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acids (18AA) 0.05%, and 4-6 μmol/L of stressor.

In a preferred technical solution of the present disclosure, the density of mesenchymal stem cells in step S-1 is 8.0×10⁶-2.0×10⁷ cells/mL, preferably 8.0×10⁶-1.0×10⁷ cells/mL.

In a preferred technical solution of the present disclosure, the mesenchymal stem cells of step S-1 are cultured in a culture medium for 3 h-5 h, preferably 3.5 h-4.5 h.

In a preferred technical solution of the present disclosure, the solvent for washing cells in step S-1 is selected from any one or a combination of physiological saline, 5% glucose solution, phosphate buffer (PBS), TBPS buffer, TBST buffer, Tris buffer, with a cell washing frequency of 2-5 times, preferably 3-4 times.

In a preferred technical solution of the present disclosure, the isolation described in step S-1 is selected from any one or a combination of centrifugation or filtration, wherein the centrifugation condition is 1000-2000 rpm*3-15 min, preferably 1200 rpm˜1500 rpm*5-10 min.

In a preferred technical solution of the present disclosure, conditions of ultrasonic treatment of step S-2 are as follows: operating at 2° C.-8° C., 25 kHZ, 360 W for 3 seconds with a gap of 1 second, and performing ultrasonic treatment for 1-5 minutes.

In a preferred technical solution of the present disclosure, the separation in step S-3 is selected from any one or a combination of 2000-8000 rpm*10-30 min centrifugation, multi-stage centrifugation, or multi-stage filtration, preferably 3000-7000 rpm*15-25 min.

In a preferred technical solution of the present disclosure, the multi-stage centrifugation in step S-3 is sequentially 3000-4000 rpm*3-5 minutes, 5000-6000 rpm*3-5 minutes, and 7000 rpm*5-8 minutes.

In a preferred technical solution of the present disclosure, the pore size of the multi-stage filtration membrane is selected from any one of 80 μm, 50 μm, 30 μm, 10 μm, or 5 μm.

In a preferred technical solution of the present disclosure, adding 25 U/mL to 30 U/mL of any one of or a combination of nuclease or omnipotent nuclease to a cell protein extract prepared in S-3, and performing enzymatic hydrolysis at 37° C.±1° C. for 20-30 minutes to obtain an enzymatic hydrolysate.

In a preferred technical solution of the present disclosure, the nuclease is selected from any one or a combination of an RNA nuclease or a DNA nuclease.

In a preferred technical solution of the present disclosure, the cell protein extract prepared in step S-3 or the protein composition prepared in step (2) is frozen, preferably at −40° C. to −20° C.

In a preferred technical solution of the present disclosure, a freeze-dried protectant is added to the cell protein extract prepared in step S-3 or the protein composition prepared in step (2), and freeze-dried to obtain a cell protein extract freeze-dried preparation or protein composition freeze-dried preparation, wherein the freeze-dried protectant is selected from any one or a combination of mannitol, sorbitol, dextran, glycerol, sucrose, trehalose, glucose, lactose, maltose, glucan, glycerol trioctanoate (HES), polyethylene glycol, ethylene glycol, phosphate, acetate, citrate, sorbitol, or starch.

In a preferred technical solution of the present disclosure, the freeze-dried preparation contains a freeze-dried protectant of 0.5-8% by a mass percentage, preferably 1-5%.

In a preferred technical solution of the present disclosure, a protein stabilizer is optionally added to the cell protein extract prepared in step S-3 or the protein composition prepared in step (2), wherein the protein stabilizer is selected from any one of albumin, zinc salt, or aluminum salt.

The preferred technical solution of the present disclosure, wherein the cell protein extract freeze-dried preparation or the protein composition freeze-dried preparation has a pH of 6-8, preferably a pH of 7-7.5.

In a preferred technical solution of the present disclosure, the molecular weight of the neural repair protein composition is 20 kDa to 250 kDa, preferably 35 kDa to 200 kDa.

The preferred technical solution of the present disclosure, wherein the protein composition in the neural repair protein composition is shown in FIG. 2.

In a preferred technical solution of the present disclosure, the freeze-dried preparation is dissolved in physiological saline or 5% glucose solution before use, and then used in any or a combination of intravenous injection, intrathecal injection, or lumbar puncture.

In a preferred technical solution of the present disclosure, the cultivation of mesenchymal stem cells or primary mesenchymal stem cells adopts the cultivation method in the field.

In a preferred technical solution of the present disclosure, the cultivation of mesenchymal stem cells comprises the following steps: adding primary mesenchymal stem cells to a passaging medium with an initial density of 5.0×10⁵-5.0×10⁶ cells/ml, and then culturing them under conditions of 37.0° C.±0.5° C. and 5%±1.0% CO₂ for 10-15 days. Every 2-3 days, after observing the yellowing of the passaging medium, half of the passaging medium is replaced, wherein the passaging medium contains DMEM/F12 medium containing 10% FBS, 100 U/ml penicillin, and 100 μg/ml streptomycin.

In a preferred technical solution of the present disclosure, the cultivation of primary mesenchymal stem cells includes the following steps:
1) cleaning and disinfecting an umbilical cord, dissecting the tissue, taking the Wharton's Jelly, cutting it into 3 mm³ small pieces, centrifuging, cleaning, collecting tissue blocks, placing them in DMEM/F12 medium containing 10% fetal bovine serum FBS, 100 μg/ml penicillin, and 100 μg/ml streptomycin, and then culturing them under conditions of 37.0° C.±0.5° C. and 5%±1.0% CO₂, replacing half of the medium every 2-3 days and culturing until cells growing out from the tissue blocks;
2) shaking and collecting low-layer cells, washing with PBS, adding 0.25% trypsin for digestion for 2-3 minutes, adding an equal volume of trypsin termination solution to stop digestion, gently blowing with a straw, centrifuging at 1200-1500 rpm/min*5-8 minutes, and collecting the cells.

The purpose of the present disclosure is to provide a preparation method for a cell protein extract having neural repair efficacy, including the following steps:
S-1: placing mesenchymal passage cells with a density of 5.0×10⁶ cells/mL to 5.0×10⁷ cells/mL in a culture medium containing DMEM/F12 40-50%, RPMI1640 40-50%, bovine serum albumin (BSA) 0.1-2%, epidermal growth factor (EGF) 1-15 μg/mL, fibroblast growth factor (FGF) 1-15 μg/mL, insulin transferrin 1-15 μg/mL, compound amino acids (18AA) 0.01-0.1%, and 2-10 μmol/L of a stressor, and then culturing the cells under conditions of 37.0° C.±0.5° C. and 5%±1.0% CO₂ for 2 to 6 hours, and then performing isolation, washing, and collecting cells, wherein the stressor is selected from any one of compounds 1-16 or a combination thereof;
S-2: dispersing the collected cells in a solvent at a density of 5.0×10⁶ cells/mL-5.0×10⁷ cells/mL, and then performing an ultrasonic treatment on the collected cells at 2° C.-8° C. to prepare a cell lysate, wherein the solvent is selected from any one or combination of physiological saline, 5% glucose solution, phosphate buffer solution (PBS), TBPS buffer, TBST buffer, or Tris buffer;
S-3: separating the cell lysate prepared in step S-2, then sequentially filtering a separated solution through 0.45 μm, 0.22 μm filter membranes, thereby obtaining the cell protein extract.

In a preferred technical solution of the present disclosure, the culture medium in step S-1 contains DMEM/F12 42-45%, RPMI1640 42-45%, bovine serum albumin (BSA) 0.5-1.5%, epidermal growth factor (EGF) 5-10 μg/mL, fibroblast growth factor (FGF) 5-10 μg/mL, insulin transferrin 5-10 μg/mL, compound amino acids (18AA) 0.02-0.05%, and 3-8 μmol/L of stressor.

In a preferred technical solution of the present disclosure, the culture medium in step S-1 contains DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acids (18AA) 0.05%, and 4-6 μmol/L of stressor.

In a preferred technical solution of the present disclosure, the density of mesenchymal stem cells in step S-1 is 8.0×10⁶-2.0×10⁷ cells/mL, preferably 8.0×10⁶-1.0×10⁷ cells/mL.

In a preferred technical solution of the present disclosure, the mesenchymal stem cells of step S-1 are cultured in a culture medium for 3 h-5 h, preferably 3.5 h-4.5 h.

In a preferred technical solution of the present disclosure, the solvent for washing cells in step S-1 is selected from any one or a combination of physiological saline, 5% glucose solution, phosphate buffer (PBS), TBPS buffer, TBST buffer, Tris buffer, with a cell washing frequency of 2-5 times, preferably 3-4 times.

In a preferred technical solution of the present disclosure, the isolation in step S-1 is selected from any one or a combination of centrifugation and filtration, wherein the centrifugation conditions are 1000-2000 rpm*3-15 min, preferably 1200 rpm˜1500 rpm*5-10 min.

In a preferred technical solution of the present disclosure, the conditions of ultrasonic treatment of step S-2 are as follows: operating at 2° C.-8° C., 25 kHZ, 360 W for 3 seconds with a gap of 1 second, and performing ultrasonic treatment for 1-5 minutes.

In a preferred technical solution of the present disclosure, the isolation in step S-3 is selected from any one or a combination of 2000-8000 rpm*10-30 min centrifugation, multi-stage centrifugation, and multi-stage filtration, preferably 3000-7000 rpm*15-25 min.

In a preferred technical solution of the present disclosure, the multi-stage centrifugation in step S-3 is sequentially 3000-4000 rpm*3-5 minutes, 5000-6000 rpm*3-5 minutes, and 7000 rpm*5-8 minutes.

In a preferred technical solution of the present disclosure, the pore size of the multi-stage filtration membrane is selected from any one of 80 μm, 50 μm, 30 μm, 10 μm, or 5 μm.

In a preferred technical solution of the present disclosure, the cell protein extract prepared in step S-3 is frozen, preferably at −40° C. to −20° C.

In a preferred technical solution of the present disclosure, the cell protein extract prepared in step S-3 is hydrolyzed by either nuclease or omnipotent nuclease before separation and purification.

In a preferred technical solution of the present disclosure, the cultivation of mesenchymal stem cells or primary mesenchymal stem cells adopts the cultivation method in the field.

In a preferred technical solution of the present disclosure, the cultivation of mesenchymal stem cells comprises the following steps: adding primary mesenchymal stem cells to a passaging medium with an initial density of 5.0×10⁵-5.0×10⁶ cells/ml, and then culturing them under conditions of 37.0° C.±0.5° C. and 5%±1.0% CO₂ for 10-15 days. Every 2-3 days, after observing the yellowing of the passaging medium, half of the passaging medium is replaced, wherein the passaging medium contains DMEM/F12 medium containing 10% FBS, 100 U/ml penicillin, and 100 μg/ml streptomycin.

In a preferred technical solution of the present disclosure, the cultivation of primary mesenchymal stem cells includes the following steps:
1) cleaning and disinfecting an umbilical cord, dissecting the tissue, taking the Wharton's Jelly, cutting it into 3 mm³ small pieces, centrifuging, cleaning, collecting tissue blocks, placing them in DMEM/F12 medium containing 10% fetal bovine serum FBS, 100 μg/ml penicillin, and 100 μg/ml streptomycin, and then culturing them under conditions of 37.0° C.±0.5° C. and 5%±1.0% CO₂, replacing half of the medium every 2-3 days and culturing until cells growing out from the tissue blocks;
2) shaking and collecting low-layer cells, washing with PBS, adding 0.25% trypsin for digestion for 2-3 minutes, adding an equal volume of trypsin termination solution to stop digestion, gently blowing with a straw, centrifuging at 1200-1500 rpm/min*5-8 minutes, and collecting the cells.

The purpose of the present disclosure is to provide a preparation method for a neural repair protein composition, including the following steps:
(1) adding 20 U/mL-35 U/mL of any one of or a combination nuclease or omnipotent nuclease to a cell protein extract of the present disclosure, and performing enzymatic hydrolysis at 37° C.±1° C. for 15-40 minutes to obtain an enzymatic hydrolysate;
(2) under conditions of 2° C.-8° C., preparing the enzymatic hydrolysate obtained in step (1) to a 5-15 mg/ml solution with an eluent, and then passing through a chromatographic column with an eluent flow rate of 0.1-1 mL/min, monitoring and collecting an eluate fraction with a UV wavelength of 280 nm, wherein the eluent solvent consists of 50 mmol/L phosphate buffer (pH 6.8) containing 300 mmol/L sodium chloride.

In a preferred technical solution of the present disclosure, the nuclease is selected from any one or a combination of an RNA nuclease or a DNA nuclease.

In a preferred technical solution of the present disclosure, adding 20 U/mL-35 U/mL of any one of or a combination of nuclease or omnipotent nuclease to a cell protein extract of the present disclosure, and performing enzymatic hydrolysis at 37° C.±1° C. for 15-40 minutes to obtain an enzymatic hydrolysate;

In a preferred technical solution of the present disclosure, the molecular weight of the neural repair protein composition is 20 kDa to 250 kDa, preferably 35 kDa to 200 kDa.

In a preferred technical solution of the present disclosure, the protein composition obtained in step (2) is frozen, preferably at −40° C. to −20° C.

In a preferred technical solution of the present disclosure, a freeze-dried protectant is added to the protein composition collected in step (2), and freeze-dried to obtain a protein composition freeze-dried preparation, wherein the freeze-dried protectant is selected from any one or a combination of mannitol, sorbitol, dextran, glycerol, sucrose, trehalose, glucose, lactose, maltose, glucan, glycerol trioctanoate (HES), polyethylene glycol, ethylene glycol, phosphate, acetate, citrate, sorbitol, or starch.

In a preferred technical solution of the present disclosure, the freeze-dried preparation contains a freeze-dried protectant of 0.5-8% by a mass percentage, preferably 1-5%.

In a preferred technical solution of the present disclosure, a protein stabilizer is optionally added to the protein composition collected in step (2), wherein the protein stabilizer is selected from any one of albumin, zinc salt, or aluminum salt.

The preferred technical solution of the present disclosure is the freeze-dried preparation has a pH of 6-8, preferably a pH of 7-7.5.

The preferred technical solution of the present disclosure, wherein the protein composition in the neural repair protein composition is shown in FIG. 2.

In a preferred technical solution of the present disclosure, the freeze-dried preparation is dissolved in physiological saline or 5% glucose solution before use, and then used in any or a combination of intravenous injection, intrathecal injection, or lumbar puncture.

Another object of the present disclosure is to provide a neural repair composition, which is composed of any one or a combination of cell protein extracts or neural repair protein compositions with neural efficacy of the present disclosure, and a pharmaceutically acceptable carrier.

The pharmaceutically acceptable dosage or type of carrier of the present disclosure depends on factors such as the physicochemical properties and content of the active ingredients in the composition, formulation type, dissolution and bioavailability of the formulation.

The composition of the present disclosure can be various dosage forms in the field, and can be prepared using formulation techniques in the field.

In a preferred technical scheme of the disclosure, the composition is selected from any kind of freeze-dried preparation, gel, nasal spray, paste, cream, emulsion, liquid dressing, injection and suppository.

In a preferred technical solution of the present disclosure, the administration method of the combination is selected from any one or a combination of intravenous injection, intrathecal injection, or lumbar puncture.

Another object of the present disclosure is to provide a cell protein extract, a combination of nerve repair proteins, or use thereof for the preparation of any product for cell repair or nerve repair, which has the effect of repairing nerve cells.

In a preferred technical scheme of the disclosure, the nerve repair is selected from any of the central nerve damage, neurodegenerative disease, stroke, brain trauma, ataxia, cerebral hemorrhage, Alzheimer's disease, Parkinson's disease, Alzheimer's disease, Alzheimer's disease, multiple sclerosis, Huntington's disease, amyotrophic lateral sclerosis, peripheral nerve damage, diabetes, memory loss or the nerve damage caused by its complications.

In a preferred technical solution of the present disclosure, the ataxia is selected from any one of cerebellar ataxia, cerebral ataxia, sensory ataxia, vestibular ataxia or their complications.

Another object of the present disclosure is to provide use of compounds 1-16 in the preparation of functional proteins having repair effects by stress-induced stem cell.

In a preferred technical solution of the present disclosure, the repair is any one of cell repair, hair follicle repair, joint repair, or nerve repair.

Unless otherwise specified, when the present disclosure relates to the percentage between liquids, the said percentage is volume/volume percentage; When the present disclosure relates to the percentage between liquid and solid, the percentage is the volume/weight percentage; When the present disclosure relates to the percentage between solid and liquid, the percentage is the weight/volume percentage; The rest are weight/weight percentage.

Unless otherwise specified, the identification of mesenchymal stem cells (MSCs) in the present disclosure refers to “Standards for the culture and quality control of umbilical cord mesenchymal stromal cells for neurorestorative clinical application”.

The present disclosure uses commercially available reagent kits to detect levels of reactive oxygen species (IOD), superoxide dismutase SOD, and malondialdehyde MDA.

Compared with prior art, the present disclosure has the following beneficial effects:

1. The disclosure scientifically screens the culture medium containing stressors to induce mesenchymal stem cells to produce functional proteins having cell repair and nerve repair effects. The obtained cell protein extracts, protein compositions or their compositions have cell repair and nerve repair effects, and can be used to repair central nerve damage, neurodegenerative diseases, stroke, brain trauma, ataxia, cerebral hemorrhage, Alzheimer's disease, Parkinson's disease, senile dementia, multiple sclerosis, Huntington's disease, amyotrophic lateral sclerosis, peripheral nerve damage, diabetes, memory loss, cerebellar ataxia, cerebral ataxia, sensory ataxia, vestibular ataxia Nerve damage caused by any disorder or its complication, with high purity, good stability, high bioavailability The advantages of safety, effectiveness, ease of production and storage and transportation.

2. The preparation method of the present disclosure has the advantages of simple operation, green environmental protection, lower cost, and suitability for industrial production.

### BRIEF DESCRIPTION OF THE DRAWINGS

FIG. 1. Electrophoretic separation results of the neural repair protein composition of the present disclosure;

FIG. 2. High performance liquid phase detection results of the neural repair protein composition of the present disclosure;

FIG. 3. Study on the repair effect of the cell protein extract of the present disclosure on skin damage caused by UVB irradiation;

FIG. 4. The effect of the cell protein extract of the present disclosure on the generation of oxides (IOD) in skin damage caused by UVB irradiation. Mean±SD (n=3). ## indicates that compared with the blank control (BC), p<0.01. * indicates that compared with the NC (negative control) group, p<0.05, ** indicates that compared with the NC (negative control) group, p<0.01;

FIG. 5. The effect of the cell protein extract of the present disclosure on the generation of superoxide dismutase SOD in skin damage caused by UVB irradiation. Mean±SD (n=3). ## indicates that compared with the blank control (BC), p<0.01. * indicates that compared with the NC (negative control) group, p<0.05, ** indicates that compared with the NC (negative control) group, p<0.01;

FIG. 6. The effect of the cell protein extract of the present disclosure on MDA levels in skin damage caused by UVB irradiation. Mean±SD (n=3). ## indicates that compared with the blank control (BC), p<0.01. * indicates that compared with the NC (negative control) group, p<0.05, ** indicates that compared with the NC (negative control) group, p<0.01;

FIG. 7. Comparison of weekly body weight between the treatment group and the model group in the study of the damage repair effect of the neural repair protein composition of the present disclosure on ischemic stroke;

FIG. 8. TTC staining results of the treatment group and model group in the study of the damage repair effect of the neural repair protein composition of the present disclosure on ischemic stroke;

FIG. 9. Immunofluorescence staining results of iba1 positive cells in the treatment group and model group in the study of the damage repair effect of the neural repair protein combination of the present disclosure on ischemic stroke;

FIG. 10. The study on the damage repair effect of the neural repair protein composition of the present disclosure on ischemic stroke in the treatment group and model group β Immunofluorescence staining results of 3Tubulin positive cells;

FIG. 11. The neural function (NDS) score results in the study of the repair effect of the neural repair protein composition of the present disclosure on ischemic stroke damage.

### DETAILED DESCRIPTION OF THE EMBODIMENTS

The following will further explain and describe the detailed content of the present disclosure in conjunction with specific embodiments, but this does not limit the scope of protection of the present disclosure.

### 1. Culture of Primary Mesenchymal Stem Cells

The cultivation of primary mesenchymal stem cells includes the following steps:
1) after cleaning and disinfecting the umbilical cord, dissecting the tissue, taking the Wharton's Jelly, cutting it into 3 mm³ small pieces, centrifuging, cleaning, collecting tissue blocks, placing them in a culture bottle, adding DMEM/F12 culture medium containing 10% fetal bovine serum FBS, 100 μg/ml penicillin, and 100 μg/ml streptomycin, and then culturing it under 37° C. and 5% CO₂ conditions to promote its adhesion. Every 2-3 days, observing the yellowing of the culture medium, replacing half of the culture medium, and culturing for 10-12 days until cells can be seen climbing out at the edge of the tissue blocks;
2) gently shaking to make the tissue blocks fall off, collecting the tissue block and lower layer cells separately, and then attaching the collected tissue blocks to the wall for culture;
3) after cleaning the collected low-layer cells with PBS, adding an appropriate amount of 0.25% trypsin and digesting for 2-3 minutes, adding an equal volume of trypsin termination solution to stop digestion. Gently blowing the bottom of the bottle with a straw, centrifuging at 1500 rpm/min*5 minutes, and collecting the cells.

### 2. Passage Culture of Primary Mesenchymal Stem Cells (Culture of Mesenchymal Stem Cells)

Passage culture of primary mesenchymal stem cells (Culture of Mesenchymal Stem Cells): primary mesenchymal stem cells were added to DMEM/F12 medium containing 10% FBS, 100 U/ml penicillin, and 100 μg/ml streptomycin at an initial density of 5.0×10⁵-5.0×10⁶ cells/ml, and then cultured under conditions of 37.0° C.±0.5° C. and 5%±1.0% CO₂ for 10-15 days, with intervals of 2-3 days. After observing the yellowing of the medium, half of the medium was replaced.

3. Reference 1 for the preparation of compounds 1-16 (New limonophyllines A-C from the stem of Atalantia monophylla and cytotoxicity against cholangiocarcinoma and HepG2 cell lines, Arch. Pharm. Res. (2018) 41:431-437).

### Example 1: Preparation of Cell Protein Extract Having Nerve Repair Effect of the Present Disclosure

The preparation method of a cell protein extract having neural repair effect of the present disclosure included the following steps:
(1) adding mesenchymal passage cells with a density of 8.0×10⁶ cells/mL to the culture medium containing DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acids (18AA) 0.05% and compound 16 with 5 μmol/L; then incubating the cells at 37° C. and 5% CO₂ for 4 hours, then centrifuging it at 1200 rpm*5 minutes, washing it with PBS three times, and collecting the cells;
(2) dispersing the collected cells in step (1) into physiological saline at a density of 1.0×10⁷ cells/mL, and performing an ultrasonic treatment on the collected cells for 3 seconds with a gap of 1 second under conditions of 2-8° C., 25 kHz, and 360 W for 2 minutes to prepare a cell lysis solution;
(3) centrifuging the cell lysis solution prepared in step (2) at 7000 rpm*20 minutes, and then sequentially filtering a separated solution through 0.45 μm and 0.22 μm filter membranes, thereby obtaining the cell protein extract. The prepared cell protein extract can be frozen and stored at −40° C. to −20° C. as needed.

### Example 2: Preparation of the Neural Repair Protein Composition of the Present Disclosure

The preparation of the neural repair protein composition of the present disclosure included the following steps:
(1) adding 25 U/mL of UCF. ME UltraNuclease to the cell protein extract prepared in Example 1, and incubating it at 37° C. for 30 minutes to obtain the enzymatic hydrolysate;
(2) under the conditions of 2° C.-8° C., preparing the enzymatic hydrolysate prepared in step (1) to a 10 mg/ml solution with an elution, then passing through a high-purity silica gel liquid chromatography protective column (WondaGuard C18, 4.6×5 mm) and a high-purity silica gel liquid chromatography preparation column (SHIMSEN Ankylo C18, 5 μm. 4.6×250 mm), with an elution flow rate of 0.1-1 ml/min, and the elution fraction with a UV wavelength of 280 nm was monitored and collected. The elution consisted of 50 mmol/L phosphate buffer (pH 6.8) containing 300 mmol/L sodium chloride.

### Example 3: Preparation of the Freeze-Dried Preparation of the Neural Repair Protein Composition of the Present Disclosure

Adding mannitol to the cell protein composition prepared in Example 2, stirring, mixing well, and then freezing dry. The resulting freeze-dried preparation contained 2.15% mannitol (m/m).

### Example 4: Preparation of Cell Protein Extract with Nerve Repair Effect of the Present Disclosure

The preparation method of a cell protein extract with neural repair effect of the present disclosure included the following steps:
(1) adding mesenchymal passage cells with a density of 1.0×10⁷ cells/mL to the culture medium containing DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acids (18AA) 0.05% and compound 13 with 8 μmol/L; then incubating it at 37° C. and 5% CO₂ for 4 hours, then centrifuging it at 1200 rpm*5 minutes, washing it with PBS three times, and collecting the cells;
(2) dispersing the collected cells in step (1) into physiological saline at a density of 2.0×10⁷ cells/mL, and performing an ultrasonic treatment on the collected cells for 3 seconds with a gap of 1 second under conditions of 2-8° C., 25 kHz, and 360 W for 2 minutes to prepare a cell lysis solution;
(3) centrifuging the cell lysis solution prepared in step (2) at 7000 rpm*20 minutes, and then sequentially filtering a separated solution through 0.45 μm and 0.22 μm filter membranes, thereby obtaining the cell protein extract.

### Example 5: Preparation of the Neural Repair Protein Composition of the Present Disclosure

The preparation of the neural repair protein composition of the present disclosure included the following steps:
(1) adding 30 U/mL of UCF. ME UltraNuclease to the cell protein extract prepared in Example 4, and incubating it at 37° C. for 30 minutes to obtain the enzymatic hydrolysate;
(2) under the conditions of 2° C.-8° C., preparing the enzymatic hydrolysate prepared in step (1) to a 10 mg/ml solution with an elution, then passing through a high-purity silica gel liquid chromatography protective column (WondaGuard C18, 4.6×5 mm) and a high-purity silica gel liquid chromatography preparation column (SHIMSEN Ankylo C18, 5 μm. 4.6×250 mm), with an elution flow rate of 0.5 ml/min, and the elution fraction with a UV wavelength of 280 nm was monitored and collected. The elution consisted of 50 mmol/L phosphate buffer (pH 6.8) containing 300 mmol/L sodium chloride.

### Example 6 Preparation of the Freeze-Dried Preparation of the Neural Repair Protein Composition of the Present Disclosure

Adding dextran to the cell protein composition prepared in Example 5, stirring, mixing well, and then freezing dry. The resulting freeze-dried preparation contained 1% dextran (m/m).

### Example 7: Preparation of Cell Protein Extract with Nerve Repair Effect of the Present Disclosure

The preparation method of a cell protein extract with neural repair effect of the present disclosure included the following steps:
(1) adding mesenchymal passage cells with a density of 9.0×10⁶ cells/mL to the culture medium containing DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acids (18AA) 0.05% and compound 14 with 5 μmol/L; then incubating the cells at 37° C. and 5% CO₂ for 4 hours, then centrifuging it at 1200 rpm*5 minutes, washing it with PBS three times, and collecting the cells;
(2) dispersing the collected cells in step (1) into physiological saline at a density of 2.0×10⁷ cells/mL, and performing an ultrasonic treatment on the collected cells for 3 seconds with a gap of 1 second under conditions of 2-8° C., 25 kHz, and 360 W for 2 minutes to prepare a cell lysis solution;
(3) centrifuging the cell lysis solution prepared in step (2) at 7000 rpm*20 minutes, and then sequentially filtering a separated solution through 0.45 μm and 0.22 μm filter membranes, thereby obtaining the cell protein extract.

### Example 8: Preparation of the Neural Repair Protein Composition of the Present Disclosure

The preparation of the neural repair protein composition of the present disclosure included the following steps:
(1) adding 20 U/mL of UCF. ME UltraNuclease to the cell protein extract prepared in Example 7, and incubating it at 37° C. for 30 minutes to obtain the enzymatic hydrolysate;
(2) under the conditions of 2° C.-8° C., preparing the enzymatic hydrolysate prepared in step (1) to a 10 mg/ml solution with an elution, then passing through a high-purity silica gel liquid chromatography protective column (WondaGuard C18, 4.6×5 mm) and a high-purity silica gel liquid chromatography preparation column (SHIMSEN Ankylo C18, 5 μm. 4.6×250 mm), with an elution flow rate of 0.1-1 ml/min, and the elution fraction with a UV wavelength of 280 nm was monitored and collected. The elution consisted of 50 mmol/L phosphate buffer (pH 6.8) containing 300 mmol/L sodium chloride.

### Example 9: Preparation of the Freeze-Dried Preparation of the Neural Repair Protein Composition of the Present Disclosure

Adding sorbitol to the cell protein composition prepared in Example 8, stirring, mixing well, and then freezing dry. The resulting freeze-dried preparation contained 5% sorbitol (m/m).

### Example 10: Preparation of Cell Protein Extract with Nerve Repair Effect of the Present Disclosure

The preparation method of a cell protein extract with neural repair effect of the present disclosure included the following steps:
(1) adding mesenchymal passage cells with a density of 1.0×10⁷ cells/mL to the culture medium containing DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acids (18AA) 0.05% and compound 15 with 6 μmol/L; then incubating the cells at 37° C. and 5% CO₂ for 4 hours, then centrifuging it at 1200 rpm*5 minutes, washing it with PBS three times, and collecting the cells;
(2) dispersing the collected cells in step (1) into physiological saline at a density of 2.0×10⁷ cells/mL, and performing an ultrasonic treatment on the collected cells for 3 seconds with a gap of 1 second under conditions of 2-8° C., 25 kHz, and 360 W for 2 minutes to prepare a cell lysis solution;
(3) centrifuging the cell lysis solution prepared in step (2) at 7000 rpm*20 minutes, and then sequentially filtering a separated solution through 0.45 μm and 0.22 μm filter membranes, thereby obtaining the cell protein extract.

### Example 11: Preparation of the Neural Repair Protein Composition of the Present Disclosure

The preparation of the neural repair protein composition of the present disclosure included the following steps:
(1) adding 30 U/mL of UCF. ME UltraNuclease to the cell protein extract prepared in Example 10, and incubating it at 37° C. for 30 minutes to obtain the enzymatic hydrolysate;
(2) under the conditions of 2° C.-8° C., preparing the enzymatic hydrolysate prepared in step (1) to a 10 mg/ml solution with an elution, then passing through a high-purity silica gel liquid chromatography protective column (WondaGuard C18, 4.6×5 mm) and a high-purity silica gel liquid chromatography preparation column (SHIMSEN Ankylo C18, 5 μm. 4.6×250 mm), with an elution flow rate of 1 ml/min, and the elution fraction with a UV wavelength of 280 nm was monitored and collected. The elution consisted of 50 mmol/L phosphate buffer (pH 6.8) containing 300 mmol/L sodium chloride.

### Example 12: Preparation of the Freeze-Dried Preparation of the Neural Repair Protein Composition of the Present Disclosure

Adding mannitol to the cell protein composition prepared in Example 11, stirring, mixing well, and freezing dry. The resulting freeze-dried preparation contained 3% mannitol (m/m).

### Example 13: Preparation of the Freeze-Dried Preparation of the Present Disclosure

Adding the required amount of mannitol to the cell protein composition prepared in Example 1, stirring, mixing well, and freezing dry. The resulting freeze-dried preparation contained 5% mannitol (m/m).

### Example 14: Molecular Weight Distribution Detection of the Neural Repair Protein Composition of the Present Disclosure

Using standard molecular weight polyacrylamide gel electrophoresis to detect the molecular weight distribution of the nerve repair protein composition of the disclosure, included the following steps:
1. selecting a glass plate with a thickness of 1.5 mm and placing it horizontally, and spreading the gel solution (15% lower layer gel, 4% separating gel) prepared according to Table 1 onto the glass plate in sequence, and insert adhesive solution the comb vertically onto the lower layer gel;

TABLE 1

| reagent | 4% separating gel | | 15% Lower layer gel | |
|---|---|---|---|---|
| ddH₂O | 6 | ml | 3.7 | ml |
| 30% Acrylamide | 1.33 | ml | 8 | ml |
| mixed solution | | | | |
| Tris | 2.5 ml (0.5M Tris pH 6.8) | | 4 ml (1.5M Tris pH 8.8) | |
| 10% SDS | 100 | μl | 160 | μl |
| 10% APS | 100 | μl | 160 | μl |
| TEMED | 10 | μl | 18 | μl |

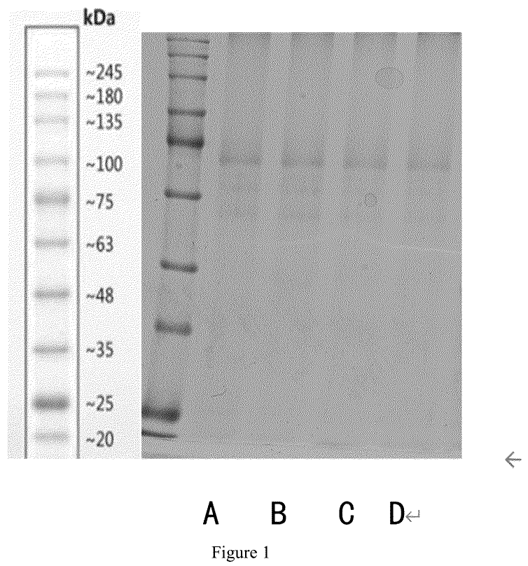

2. After thawing the frozen Marker (20 KDa-245 KDa) stored at −40° C., preparing 10 μg/μl samples of the Marker and the freeze-dried powder of the nerve repair protein composition of Embodiments 3, 6, 9 and 12 with PBS, adding 20 μl samples to the sample loading hole, performing electrophoresis at 60-80V until clear bands appeared, adjusting the voltage to 100-120 V until the Marker was completely separated, placing the PAGE gel in the Coomassie brilliant blue dye solution, and stopped dyeing when clear bands appeared on the gel. After cleaning with pure water, decolorizing with 10% acetic acid solution, and stopped decolorizing when the gel became transparent. The results were shown in FIG. 1, where band A was Example 3, band B was Example 6, band C was Example 9, and band D was Example 12.

### Example 15: High Performance Liquid Chromatography Detection of the Neural Repair Protein Composition of the Present Disclosure

Dissolving the freeze-dried powder of the nerve repair protein composition in deionized water in Example 3 and preparing it into a 10 mg/ml test solution.

Chromatographic column: SHIMSEN Ankylo (300 mm*4.6 mm. D., 3 μm; P/N: 380-01215-05) Shimadzu

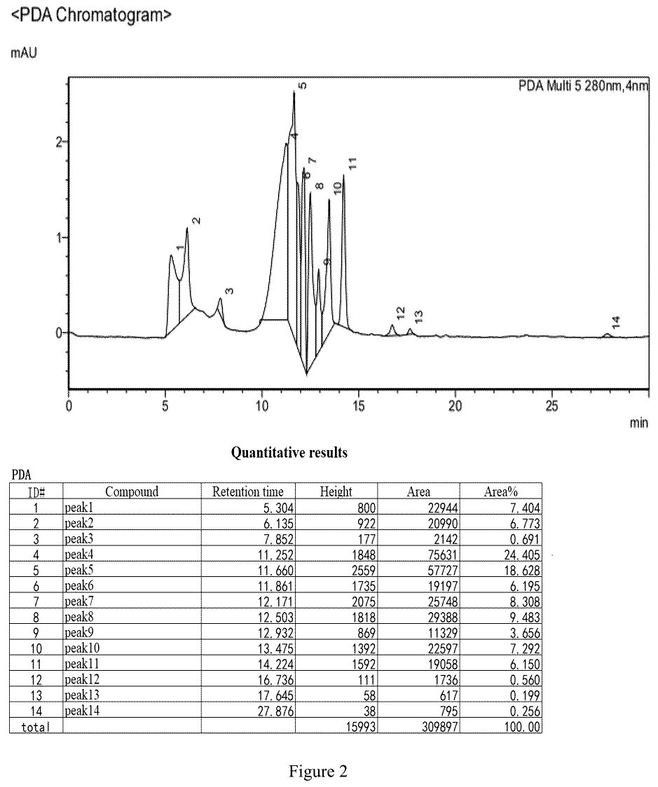

Mobile phase: 50 mmol/L phosphate buffer (pH=6.8), containing 300 mmol/L sodium chloride; Flow rate: 0.3 mL/min; Injection volume: 10 μl; Column temperature: 25° C.; Detection wavelength: 280 nm; Equal elution, collecting for 30 minutes. The results were shown in FIG. 2.

### Experimental Example 1: Study on the Repair Effect of Cell Protein Extracts of the Disclosure on UVB Radiation Damaged Skin

3D skin model: EpiKutis®, purchased from EpiKutis.

Model culture medium: DMEM basic culture medium.

VE working solution: Taking 0.5 g of VE stock solution and dissolving it in 10 mL of anhydrous ethanol to prepare a 5% mother solution; taking 100 μL of 5% mother solution and dissolving it in 10 mL of model culture medium to prepare 0.05% VE working solution.

Adding 0.9 mL of model culture medium to the 6-well plate, transferring the 3D skin model to the 6-well plate, and labeling the test number.

Blank control (BC): The skin model was left untreated and incubated in a CO₂ incubator (37° C., 5% CO₂) for 48 hours;

Negative control (NC): After irradiating the surface of the skin model with a UVB dose of 600 mJ/cm², incubating it in a CO₂ incubator (37° C., 5% CO₂) for 24 hours; adding 25 μL of 0.2% SLS working solution, incubating it in a CO₂ incubator (37° C., 5% CO₂) for 24 hours;

Positive control (PC): After irradiating the surface of the skin model with a UVB dose of 600 mJ/cm², incubating it in a CO₂ incubator (37° C., 5% CO₂) for 24 hours; adding 25 μL of 0.05% VE working solution, incubating it in a CO₂ incubator (37° C., 5% CO₂) for 24 hours;

Experimental group: After irradiating the surface of the skin model with a UVB dose of 600 mJ/cm², incubating it in a CO₂ incubator (37° C., 5% CO₂) for 24 hours; adding 25 μL of 1% cell protein extract solution (freeze-dried cell protein extract powder in Example 13 was prepared into a 1% solution with physiological saline), and then incubating it in a CO₂ incubator (37° C., 5% CO₂) for 24 hours.

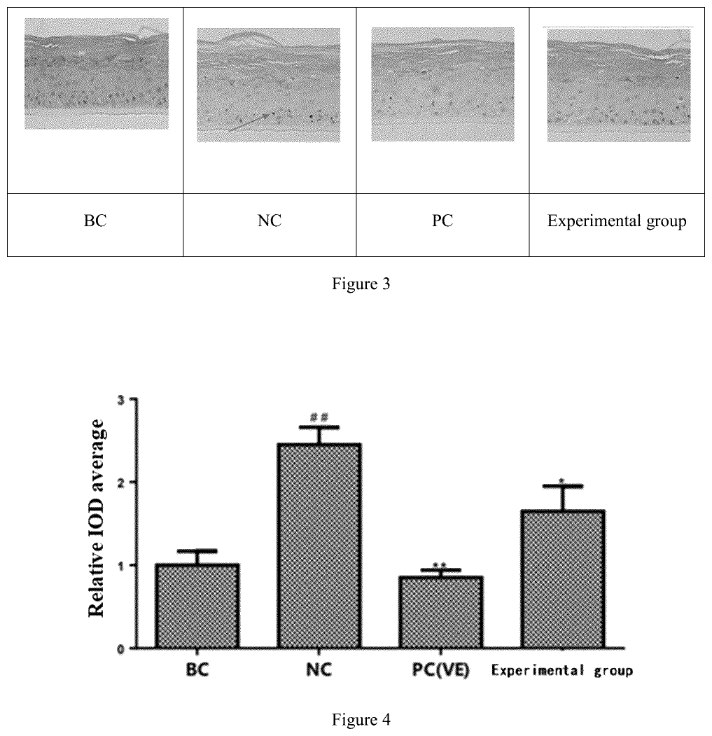

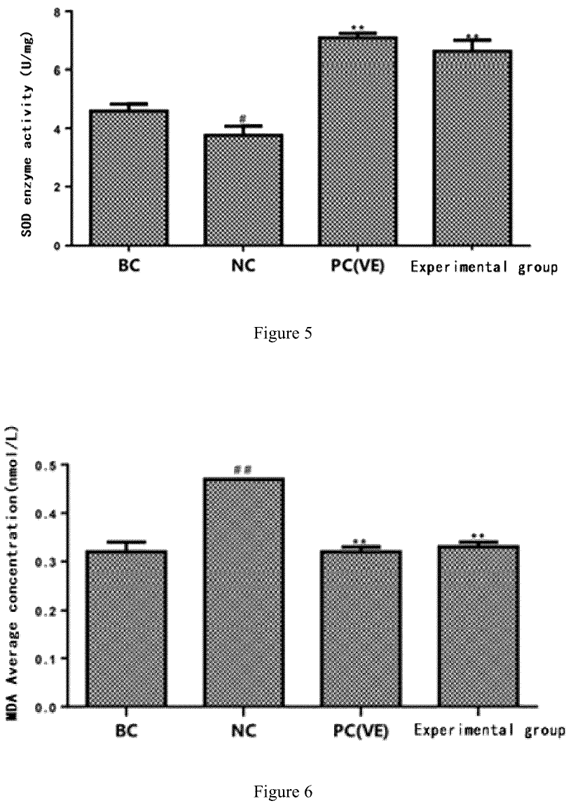

After 48 hours of incubation, washing and removing residual liquid inside and outside of the skin model with PBS solution. After 24 hours of fixation with 4% paraformaldehyde, loop cutting the model and observing after H&E staining. The results were shown in FIG. 3. Detecting the levels of reactive oxygen species (ROS), superoxide dismutase, and malondialdehyde (MDA) in UVB radiation damaged skin were as shown in FIGS. 4 to 6. The cell protein extract of the present disclosure had a significant repairing effect on skin damage caused by UVB radiation.

### Experimental Example 2: Investigation of the Repair Effect of the Neural Repair Protein Composition of the Disclosure on Ischemic Stroke Damage

Selecting 75 SPF grade male SD rats, aged 6-7 weeks. After the experimental animals were fed with regular feed for one week to adapt, all animals underwent cerebral ischemia-reperfusion surgery to construct a stroke model. Once the reperfusion was completed for the experimental animal, they were immediately subjected to neurological function scoring after regaining consciousness from anesthesia. The experimental rats fasted for one night on the day before the experiment, were anesthetized and fixed in a supine position on the surgical board. A longitudinal incision of about 3 cm was made in the neck, exposing and bluntly freeing the common carotid artery (CCA), external carotid artery (ECA), and internal carotid artery (ICA). A live knot was tied at the proximal end of the common carotid artery (CCA) to temporarily block blood supply. The external carotid artery (ECA) was tied in double knots, and a small incision was made in the middle of the double knots using a microscope. The external carotid artery (ECA) was severed from the middle of the double knots, and the direction of the suture was adjusted to enter the internal carotid artery (ICA). The internal carotid artery (ICA) and carotid artery (ICA) were tied in double knots, and a thread was inserted through a small incision. The bifurcation of the external artery (ECA) was the reference position, and the thread plug was inserted to the preset scale, feeling resistance and indicating that the head of the thread plug had reached the middle cerebral artery (MCA). The filament was then secured with a knot. After 60 minutes of ischemia, the filament was removed, and the slipknot on the common carotid artery was loosened to complete the procedure.

62 experimental animals survived after surgery. According to the scoring results, the experimental animals were divided into a model group (30 animals) and a treatment group (32 animals), both of which were injected into the lateral ventricle three times, including immediate administration after cerebral ischemia-reperfusion, administration one week after surgery, and administration two weeks after surgery. The treatment group (the freeze-dried powder of the nerve repair protein combination in Example 3 was dissolved in physiological saline, with a dosage of 6.77 mgf/kg and a dosage volume of 20 μl), while the model group was given the same volume of physiological saline to the corresponding lateral ventricle. Three days after surgery, two animals from each group were selected for TTC staining; four weeks after surgery, 15 animals were taken from the model group and 16 animals were taken from the treatment group. One experimental animal was selected from each group for TTC staining; eight weeks after surgery, 13 animals were taken from the model group and 14 animals were taken from the treatment group. One experimental animal was selected from each group for TTC staining.

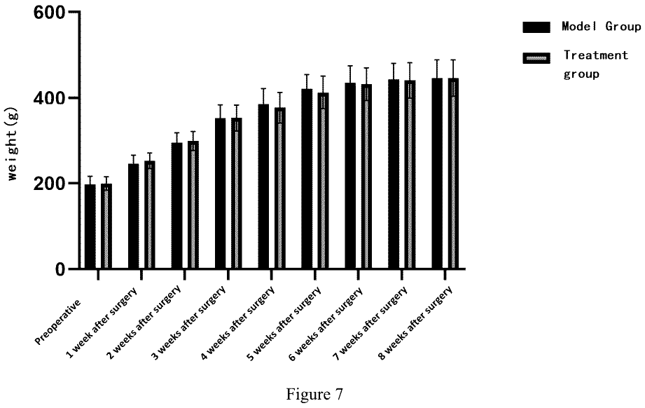

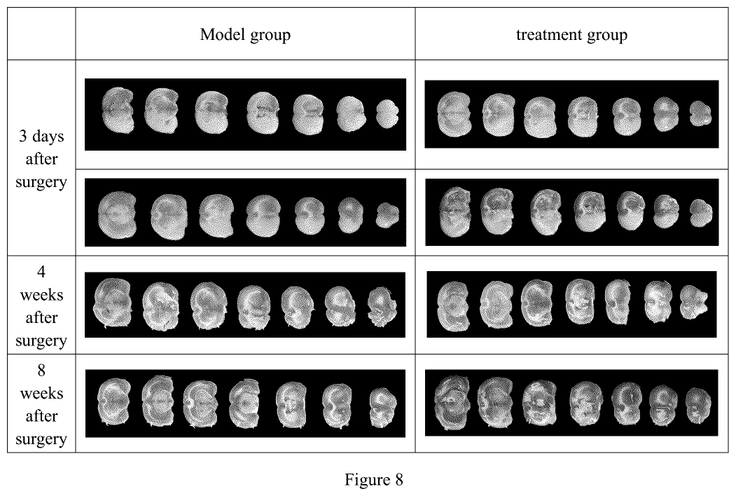

During the experiment, observing the experimental animals once a week, including their mortality, mental state, behavioral activity, onset of disease, respiration, secretions, feces, diet, and water intake. Measuring the weight of all experimental animals once a week, with the results shown in FIG. 7. During the experiment, there was no significant difference in weight between the treatment group and the model group.

Evaluating the consistency of the stroke model 3 days, 4 weeks, and 8 weeks after surgery. After blood collection, immediately inserting a catheter from the left ventricle, quickly irrigating with physiological saline for 2 minutes, cutting the head and taking the brain for TTC staining. Cutting the brain into 5-7 coronal sections from front to back, with a thickness of about 1.8 mm. Staining with 1% TTC at 37° C. for 10 minutes, fixing with tissue fixative, scanning with a scanner, and analyzing with analysis software. It reacted with dehydrogenase in normal tissues and appeared red, while dehydrogenase activity in ischemic tissues decreased, preventing the reaction, so there was no change and it appeared pale. The results were shown in FIG. 8.

Using a grip strength meter to evaluate the muscle strength of the animal before and one week after surgery. The whole body endurance of the experimental animals before and 1 week after surgery was measured using a rotary fatigue tester, with a rotational speed of 12 r/min, and three consecutive experiments were conducted for 3 minutes each time, including the mean values of the falling speed, running duration, and distance traveled. The results were shown in Table 2. The treatment group had higher forelimb grip, pre fall speed, travel distance, and duration than the model group one week after surgery (P<0.01).

TABLE 2

| | | Model Group | Treatment group |
|---|---|---|---|
| testing index | dose(mg/kg) | — | 6.77 |
| | number | 28 | 30 |
| Forelimb | Preoperative | 347.75 ± 81.12 | 346.17 ± 81.96 |
| grip(g) | 1 week after | 338.18 ± 125.40 | 435.71 ± 188.85 |
| | surgery | | |
| Speed before | Preoperative | 11.95 ± 0.25 | 11.94 ± 0.27 |
| falling(m/s) | 1 week after | 11.68 ± 0.69 | 11.97 ± 0.13** |
| | surgery | | |
| Travel | Preoperative | 1.69 ± 1.01 | 1.79 ± 1.33 |
| distance(m) | 1 week after | 0.91 ± 0.81 | 1.78 ± 1.25** |
| | surgery | | |
| Duration(s) | Preoperative | 110.43 ± 59.30 | 99.62 ± 70.33 |
| | 1 week after | 49.99 ± 41.54 | 101.26 ± 64.64** |
| | surgery | | |

Note:
Compared with the model group, * represents P < 0.05, and ** represents p < 0.01.

Evaluation of learning and memory function and activity of animals in the water maze test 4 weeks and 8 weeks after surgery: acquired training experiments were conducted on the first four days, and platform exploration experiments were conducted on the fifth day. Acquired training experimental method: First, expose the platform to the water surface and allow rats to see the platform. Put the rat into the swimming pool again, and if the rat swims directly to the platform without any difficulty, it indicates that its swimming ability and vision are normal, and the experiment can begin. Install the platform 5 mm below the water surface in the second quadrant, with the rat facing away from the pool wall and placed in the water. The placement position is randomly selected as one of the starting positions of quadrants 1, 2, 3, and 4, with each quadrant placed once for a total of 4 times. Record the time when the rat found the underwater platform (to avoid the ambush period). If the escape latency exceeds 60 seconds, guide the rat to the platform, let it stay on the platform for 10 seconds, then move the rat away and dry it. If necessary, bake the rats under a 150 W incandescent lamp for 5 minutes and put them back into the cage. Platform exploration experimental method: After the completion of acquired training, the second quadrant of the platform is removed the next day. At the starting position of the fourth quadrant, the rat is placed in the water with its head facing the pool wall. After the experiment, the number of platform crossings in the acquired experiment was analyzed, and the results were shown in Table 3. The number of times the treatment group crossed the platform at 4 weeks and 8 weeks after surgery was higher than that of the model group (P<0.01).

TABLE 3

| | | Dose | | 4 weeks | | 8 weeks |
|---|---|---|---|---|---|---|
| testing index | group | (mg/kg) | number | after surgery | number | after surgery |
| number of | Model | — | 28 | 1.05 ± 1.00 | 13 | 2.92 ± 1.31 |
| times of | group | | | | | |
| crossing the | Treatment | 6.77 | 30 | 2.63 ± 1.17** | 14 | 4.60 ± 1.07** |
| platform | group | | | | | |
| (number of | | | | | | |
| times) | | | | | | |

Note:
Compared with the model group, * represents P < 0.05, and **represents p < 0.01.

Pathological examination: Tissue samples were taken with a thickness of 3 mm, dehydrated with gradient alcohol for 70%, 80%, 95%, and 100% for 30 minutes each, in two bottles of xylene for 20 minutes each, immersed in paraffin for 30 minutes each, embedded, sliced into 4 microns, baked, and stained. The results were shown in Tables 4 to 5. The percentage of atrophy and calcification area on the healthy side relative to the injured side in the treatment group were significantly lower than those in the model group at 8 weeks after surgery (P<0.01).

TABLE 4

| | | | | | | Atrophy |
|---|---|---|---|---|---|---|
| | | dose | | Healthy | Damaged | percentage |
| testing index | group | (mg/kg) | number | side | side | % |
| Hippocampus | Model | — | 5 | 5.80 ± 1.12 | 4.09 ± 1.45 | 28.57 ± 23.55 |
| area mm² | group | | | | | |
| (8 weeks after | Treatment | 6.77 | 6 | 4.89 ± 2.05 | 4.52 ± 1.97 | 2.98 ± 29.93** |
| surgery) | group | | | | | |

Note:
Compared with the model group, * represents P < 0.05, and **represents p < 0.01.

TABLE 5

| | | dose | | 8 weeks after |
|---|---|---|---|---|
| testing index | group | (mg/kg) | number | surgery |
| Calcification | Model group | — | 5 | 0.66 ± 0.15 |
| area mm² | Treatment group | 6.77 | 6 | 0.11 ± 0.06* |

Note:
Compared with the model group, * represents P < 0.05, and **represents p < 0.01.

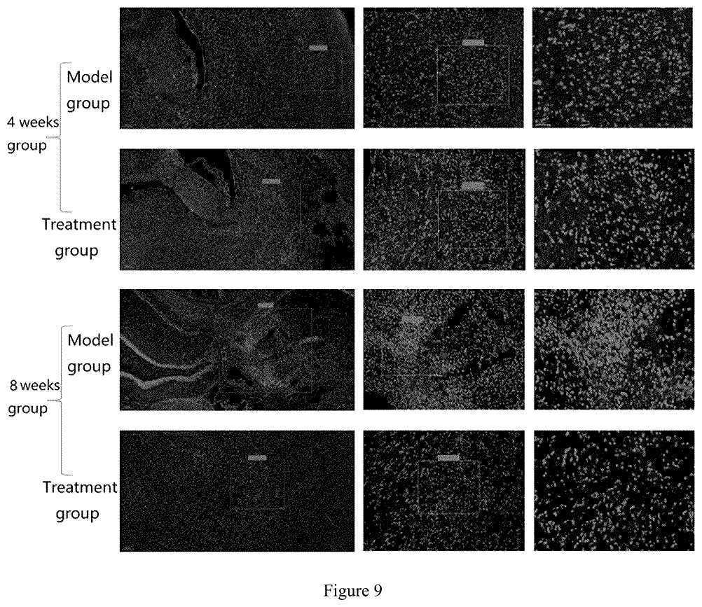

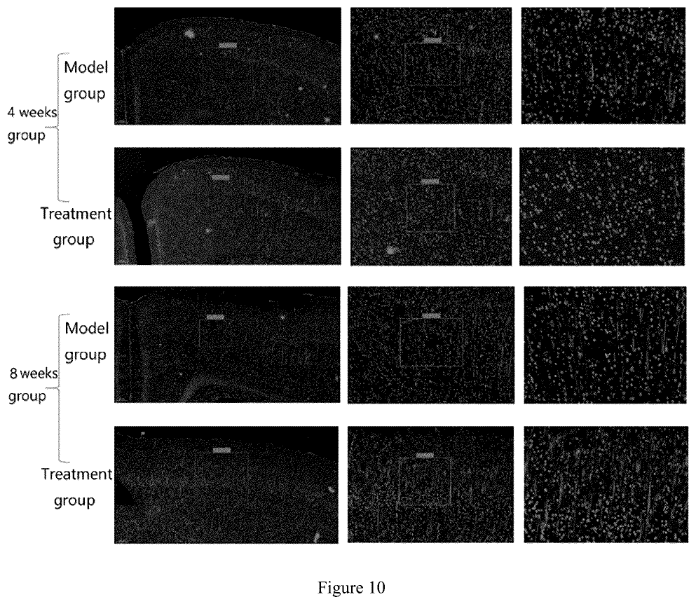

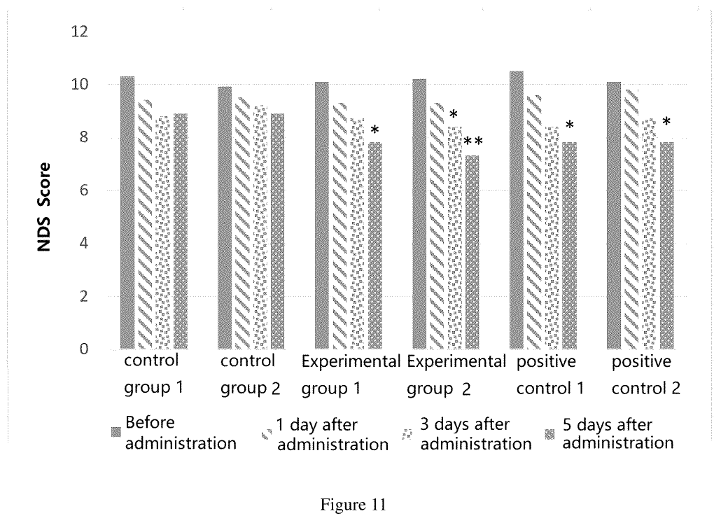

Immunofluorescence staining results (Table 6, FIG. 9-FIG. 10). The area of iba1 positive cells in the treatment group was significantly lower than that in the model group at 4 weeks and 8 weeks after surgery (p<0.05); the number of Tubulin positive cells in the 3 groups was significantly higher than that in the model group (p<0.01) 8 weeks after surgery in the treatment group β.

TABLE 6

| | | | | | β3 Tubulin Number of | |
|---|---|---|---|---|---|---|
| | | | iba1 Positive cell area (μm²) | | positive cells | |
| | dose | | 4 weeks after | 8 weeks after | 4 weeks after | 8 weeks after |
| group | (mg/kg) | number | surgery | surgery | surgery | surgery |
| Model | — | 6 | 47872.4 ± | 45459.2 ± | 69 ± | 101 ± |
| group | | | 21509.13746 | 67519.0911 | 45 | 25 |
| Treatment | 6.77 | 6 | 20822.6 ± | 12115.8 ± | 102 ± | 200 ± |
| group | | | 13412.2764* | 6290.645* | 13 | 55** |

Note:
Compared with the model group, *represents P < 0.05, and **represents p < 0.01.

### Experimental Example 3: Study on the Damage Repair Effect of the Neurorepair Protein Composition of the Disclosure on Ischemic Stroke

Selecting 80 SPF grade SD male rats weighing 220-240 g. Experimental animals were free to feed and drink water.

The experimental animals were anesthetized with isoflurane gas and maintained. Fixing in a supine position, cutting open the skin along the midline of the neck, exposing the right common carotid artery, carefully separating the nerves and fascia around the bifurcation of the common carotid artery to the blood vessels at the bottom of the skull, separating the branches of the external carotid artery, including the occipital artery, superior thyroid artery, lingual artery, and maxillary artery, ligating and cutting them off. Introducing 3 #nylon thread (diameter 0.25 mm) from the distal end of the external carotid artery into the internal carotid artery and inserting it into the Willis ring of the middle cerebral artery to effectively block the middle cerebral artery. The length of the inserted nylon thread was 18-20 mm from the bifurcation of the common carotid artery. 2 hours after MCAO, carefully removing the nylon thread from the lumen of the internal carotid artery to reperfusion the internal carotid artery. After the animal was fully awake, performing a neurological function score (NDS, see Table 7). Animal score>8 indicated successful modeling.

TABLE 7
Neurological function scoring criteria

| Project | performance | score |
|---|---|---|
| exercise test | 1) Lift the mouse tail | |
| | Forelimb curvature | 1 |
| | Bending of hind limbs | 1 |
| | Head up within 30 seconds > 10° (perpendicular to the axis) | 1 |
| | 2) Placing the mouse on the floor to walk (normal = 0; | — |
| | maximum value = 3) | |
| | Normal walking | 0 |
| | Unable to go straight | 1 |
| | Rotate towards the hemiplegic side | 2 |
| | Inverted hemiplegic side | 3 |
| Horizontal bar | Able to balance and maintain a stable posture | 0 |
| balance test | Grasp the side of the crossbar | 1 |
| | Able to hold onto the crossbar, but one limb falls off the | 2 |
| | crossbar, | |
| | Able to hold onto the crossbar, but two limbs fall off the | 3 |
| | crossbar, | |
| | Or rotate on the crossbar for more than 60 seconds | |
| | Attempted to balance on the crossbar (>40 s) but fell off | 4 |
| | Attempted to balance on the crossbar (>20 s) but fell off | 5 |
| | Falling off, unable to balance, or unwilling to struggle to hang | 6 |
| | on the crossbar | |
| Lack of reflex | Auricular reflex (shaking head when in contact with ear canal | 1 |
| or abnormal | opening) | |
| movement | Corneal reflex (blinking when lightly touched with cotton) | 1 |
| | Panic reflex (the brief sound of tearing paper produces a | 1 |
| | motion response) | |
| | Epilepsy, myoclonus, muscle tone disorders | 1 |
| Total | — | 16 |

60 rats that were successfully modeled were randomly divided into 6 groups based on their scores, with 10 rats in each group. The administration method was shown in Table 8. The experimental animals were administered intravenously after reperfusion.

TABLE 8

| | | Dosage | | |
|---|---|---|---|---|
| | Experimental | administered | Dosage | Administration |
| group | drug | (mg/kg) | volume | method |
| Control | physiological | — | 10 mL/kg | intravenous injection |
| group 1 | saline | | | 30 min |
| Control | mannitol | 3.9 | 10 mL/kg | intravenous injection |
| group 2 | | | | 30 min |
| Experimental | Example 3 | 6 | 10 mL/kg | intravenous injection |
| group 1 | | | | 30 min |
| Experimental | | 18 | 10 mL/kg | intravenous injection |
| Group 2 | | | | 30 min |
| Positive | Butylphthalide | 5 | 10 mL/kg | intravenous injection |
| control 1 | | | | 30 min |
| Positive | Yidalafeng Right | 6 | 10 mL/kg | intravenous injection |
| control 2 | Kanchun | | | 30 min |

The experimental animals were evaluated for neurological function using a blind method before and 1 day, 3 days, and 5 days after administration. The experimental data was expressed as mean±standard deviation (Mean±SEM) and statistically analyzed using SPSS 25.0 software.

Perform one-way ANOVA with equal variance, and use LSD test for pairwise comparison when there is a significant difference. Perform Kruskal Wallis non parametric test for uneven variance and compare pairwise. The results were shown in FIG. 11. Compared with before administration, experimental group 1 showed a similar repair effect on stroke nerve damage as positive control 1 and positive control 2 5 days after administration (P<0.05). Compared with before administration, experimental group 2 showed significant nerve damage repair effect (P<0.05) at 3 days after administration. Compared with before administration, experimental group 2 showed a more significant repair effect on stroke nerve damage 5 days after administration compared to positive control 1 and positive control 2 (P<0.01).

The above description of the specific embodiments of the present disclosure does not limit the present disclosure. Those skilled in the art may make various changes or deformations based on the present disclosure, as long as they do not deviate from the spirit of the present disclosure, they should fall within the scope of protection of the claims of the present disclosure.

What is claimed is:

1. A neural repair protein composition, wherein the preparation thereof comprising the following steps:
(1) adding 20 U/mL-35 U/mL of any one of or a combination of nuclease or omnipotent nuclease to a cell protein extract, and performing enzymatic hydrolysis at 37° C.±1° C. for 15-40 minutes to obtain an enzymatic hydrolysate;
(2) under conditions of 2° C.-8° C., preparing the enzymatic hydrolysate obtained in step (1) to a 5-15 mg/ml solution with an eluent, and then passing through a chromatographic column with an eluent flow rate of 0.1-1 mL/min, monitoring and collecting an eluate fraction with a UV wavelength of 280 nm, wherein the eluent consists of 50 mmol/L phosphate buffer (pH 6.8) containing 300 mmol/L sodium chloride.

2. The protein composition as claimed in claim 1, wherein the preparation of the protein extract comprises the following steps:
S-1: placing mesenchymal passage cells with a density of 5.0×10⁶ cells/mL to 1.0×10⁷ cells/mL in a culture medium containing DMEM/F12 40-50%, RPMI1640 40-50%, bovine serum albumin (BSA) 0.1-2%, epidermal growth factor (EGF) 1-15 μg/mL, fibroblast growth factor (FGF) 1-15 μg/mL, insulin transferrin 1-15 μg/mL, compound amino acids (18AA) 0.01-0.1%, and 2-10 μmol/L of a stressor, and then culturing the cells under conditions of 37.0° C.±0.5° C. and 5%±1.0% CO₂ for 2 to 6 hours, and then performing isolation, washing, and collecting cells, wherein the stressor is selected from any one of compounds 1-16 or a combination thereof;

| Compounds | General formula | Substituents |
|---|---|---|
| 1 2 3 | 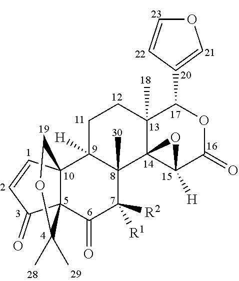 | R¹ = OCH₃, R² = OH R¹ = R² = OH R¹ = OH, R² = H |
| 4 5 | 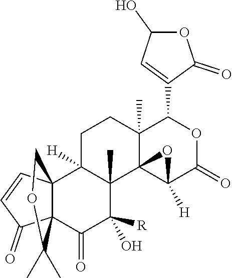 | R = H R = OH |
| 6 7 8 9 10 11 12 | 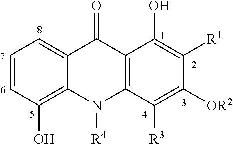 | R¹ = prenyl, R² = H, R₃ = prenyl, R⁴ = CH₃ R¹ = prenyl, R² = H, R₃ = prenyl, R⁴ = H R¹ = H, R² = H, R₃ = OCH₃, R⁴ = CH₃ R¹ = H, R² = CH₃, R₃ = OCH₃, R⁴ = CH₃ R¹ = OCH₃, R² = H, R₃ = H, R⁴ = CH₃ R¹ = OCH₃, R² = H, R₃ = OCH₃, R⁴ = CH₃ R¹ = prenyl, R² = CH₃, R₃ = H, R⁴ = H |
| 13 14 | 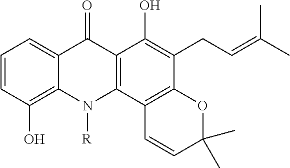 | R = H R = CH₃ |
| 15 | 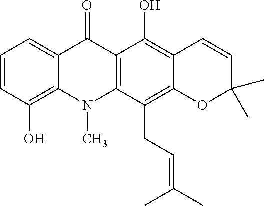 [SMILES: CC(C)=CCc1c2c(c(O)c3c(=O)c4cccc(O)c4n(C)c13)C=CC(C)(C)O2] | |
| 16 | 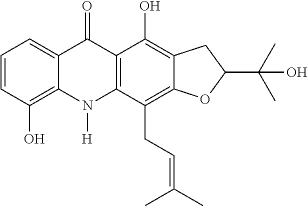 [SMILES: CC(C)=CCc1c2c(c(O)c3c(=O)c4cccc(O)c4[nH]c13)CC(C(C)(C)O)O2] | |

S-2: dispersing the collected cells in a solvent at a density of 5.0×10⁶ cells/mL-5.0×10⁷ cells/mL, and then performing an ultrasonic treatment on the collected cells at 2° C.-8° C. to prepare a cell lysate, wherein the solvent is selected from any one or a combination of physiological saline, 5% glucose solution, phosphate buffer solution (PBS), TBPS buffer, TBST buffer, or Tris buffer;
S-3: separating the cell lysate prepared in step S-2, then sequentially filtering a separated solution through 0.45 μm, 0.22 μm filter membranes, thereby obtaining the cell protein extract.

3. The protein composition as claimed in claim 2, wherein the culture medium in step S-1 contains DMEM/F12 42-45%, RPM I1640. 42-45%, bovine serum protein (BSA) 0.5-1.5%, epidermal growth factor (EGF) 5-10 μg/mL, fibroblast growth factor (FGF) 5-10 μg/mL, insulin transferrin 5-10 μg/mL, compound amino acids (18AA) 0.02-0.05% and 3-8 μmol/L of stressor.

4. The protein composition as claimed in claim 3, wherein the culture medium in step S-1 contains DMEM/F12 45%, RPMI1640 45%, bovine serum albumin (BSA) 0.5%, epidermal growth factor (EGF) 10 μg/mL, fibroblast growth factor (FGF) 10 μg/mL, insulin transferrin 10 μg/mL, compound amino acids (18AA) 0.05%, and 4-6 μMol/L of stressor.

5. The protein composition as claimed in claim 1, wherein the molecular weight of the neural repair protein composition is 20 kDa to 250 kDa, preferably 35 kDa to 200 kDa.

6. The protein composition as claimed in claim 1, wherein adding a freeze-dried protectant to the protein composition prepared in step (2) to obtain a protein composition freeze-dried preparation, wherein the freeze-dried protectant is selected from any one or a combination of mannitol, sorbitol, dextran, trehalose, glycerol, sucrose, glucose, lactose, maltose, glucan, glycerol trioctanoate, polyethylene glycol, ethylene glycol, phosphate, acetate, citrate, sorbitol, or starch.

7. The protein composition as claimed in claim 6, wherein the freeze-dried preparation comprises a freeze-dried protectant of 0.5-8% by a mass percentage, preferably 1-5%.

8. A neural repair composition comprising a neural repair protein composition as claimed in claim 1 and a pharmaceutically acceptable carrier.

9. Use of the neural repair protein composition as claimed in claim 1 in the preparation of any product for cell repair or neural repair.

10. Use of compounds 1-16 in the preparation of functional proteins having repair effects by stress-induced stem cell.

11. Use of the neural repair composition as claimed in claim 8 in the preparation of any product for cell repair or neural repair.
